(19) United States
(12) Patent Application Publication
(10) Pub. No.: US 2025/0073065 A1
(43) Pub. Date: Mar. 6, 2025

(54) SETUP ROBOT FOR FACILITATING OPHTHALMIC SURGERY

(71) Applicant: Alcon Inc., Fribourg (CH)

(72) Inventors: Patrick TERRY, Goleta, CA (US); Sumit PALIWAL, Irvine, CA (US); Mark Andrew ZIELKE, Lake Forest, CA (US); Lance NOLLER, Lake Forest, CA (US); Nanhong LOU, Southlake, TX (US)

(21) Appl. No.: 18/816,174

(22) Filed: Aug. 27, 2024

Related U.S. Application Data
(60) Provisional application No. 63/579,265, filed on Aug. 28, 2023.

(51) Int. Cl. A61F 9/007; A61B 34/30

(52) U.S. Cl. CPC A61F 9/007; A61B 34/30

(57) ABSTRACT

A system includes an actuator, one or more items of diagnostic equipment configured to perform ophthalmic measurement, and one or more items of treatment equipment configured to facilitate performance of an ophthalmic treatment with respect to an eye of a patient. A controller is coupled to the actuator and is configured to cause the actuator to transfer the one or more items of diagnostic equipment and the one or more items of treatment equipment into and out of a region in front of an eye of the patient. The ophthalmic treatment may be a LASIK or SMILE treatment using refractive error and/or eye geometry measured using the diagnostic equipment.

### CROSS-REFERENCE TO RELATED APPLICATIONS

This application claims priority to U.S. Provisional Application No. 63/579,265, filed on Aug. 28, 2023, which is hereby incorporated by reference in its entirety.

### BACKGROUND

The present disclosure relates generally to performing ophthalmic surgery.

Common ophthalmic surgical treatments include cataract surgery, glaucoma treatments, retinal membrane peeling, vitrectomy, and retinal reattachment. The structures of the eye are extremely small and delicate. Ophthalmic surgery is therefore extremely sophisticated and becoming an ophthalmic surgeon requires many years of training. The time of an ophthalmic surgeon spent in the operating room is therefore a very valuable resource.

It would be an advancement in the art to reduce the demands on the time of an ophthalmic surgeon when providing ophthalmic surgical treatments.

### SUMMARY

In certain embodiments, a system includes one or more patient stations configured to facilitate performance of ophthalmic treatments. The system includes a setup robot having the one or more patient stations in a range of motion thereof. A controller is coupled to the setup robot and configured to cause the setup robot to prepare the one or more patient stations for the ophthalmic treatments.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is schematic diagram of an operating environment including a setup robot in accordance with certain embodiments.

FIG. 1B illustrates a setup robot including a robotic arm mounted to a guide rail in accordance with certain embodiments.

FIG. 1C is a diagram illustrating a device for extracting a tool from a package in accordance with certain embodiments.

FIG. 1D illustrates a tray holding supplies for an ophthalmic treatment in accordance with certain embodiments.

FIG. 1E illustrates components for operating a setup robot in accordance with certain embodiments.

FIG. 2A is schematic diagram of an alternative operating environment including a setup robot in accordance with certain embodiments.

FIG. 2B illustrates a setup robot with ground engaging members in accordance with certain embodiments.

FIG. 2C illustrates components for operating a setup robot in accordance with certain embodiments.

FIG. 3 is a process flow diagram of a method for operating a setup robot in accordance with certain embodiments.

FIG. 4 illustrates the use of a setup robot to pass objects to a surgeon during an ophthalmic treatment in accordance with certain embodiments.

FIG. 5 illustrates the use of a setup robot to immobilize instruments during an ophthalmic treatment in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

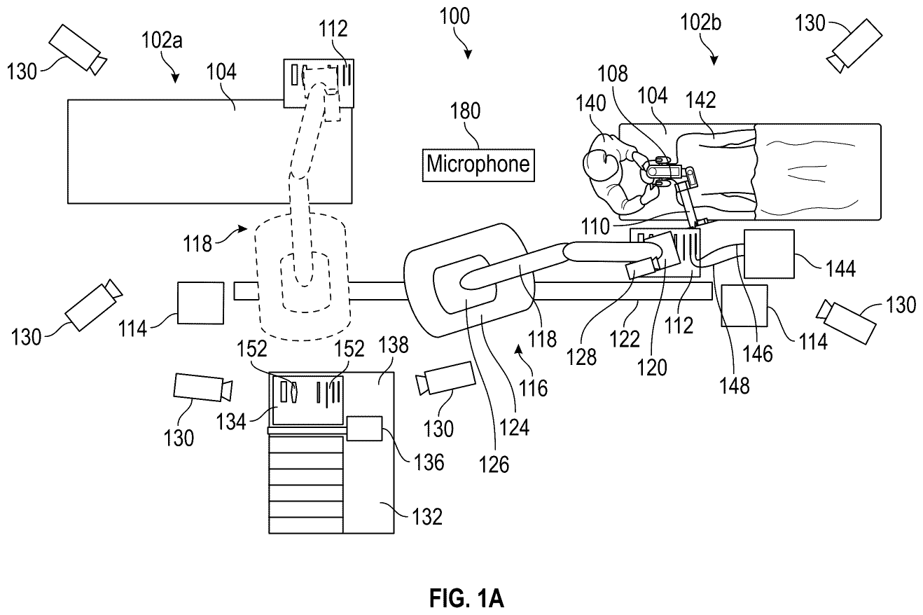

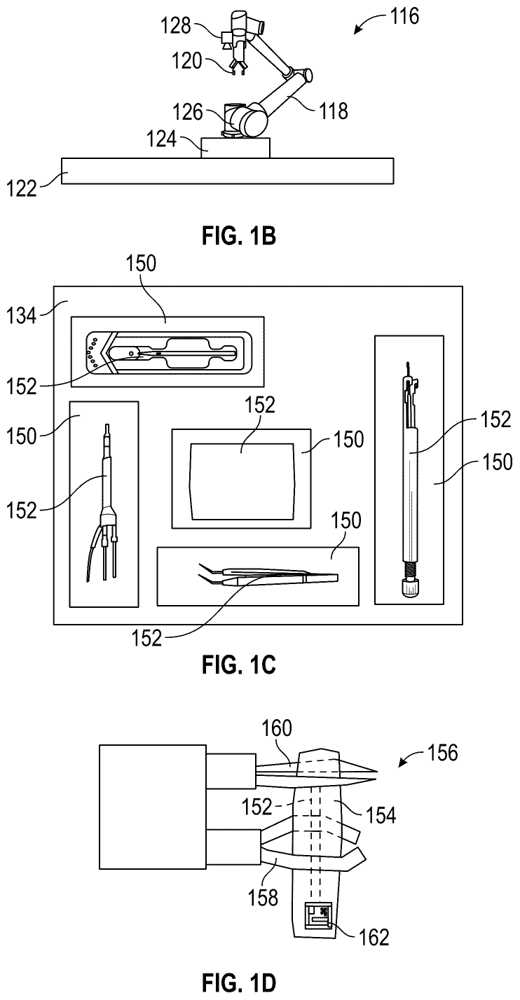

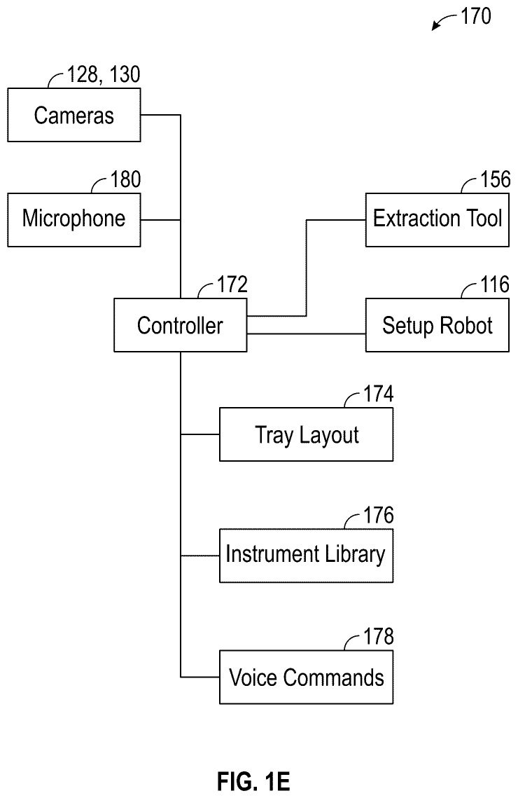

Referring to FIGS. 1A and 1B, an operating environment 100 includes two or more stations 102a, 102b, each including a patient support 104 for supporting a patient 106, such as a bed, chair, or other type of support. Although the system and methods disclosed herein are advantageously used with multiple stations 102a, 102b, a single station 102a may also be used.

Each station 102a, 102b may further include other equipment, such as a surgical microscope 108 mounted to an adjustable support 110. The surgical microscope may be implemented as the NGENUITY 3D VISUALIZATION SYSTEM provided by Alcon Inc. of Fort Worth Texas. In some embodiment, a single surgical microscope 108 is used with the adjustable support 110 facilitating movement of the surgical microscope 108 between stations 102a, 102b.

Each station 102a, 102b may further include a table 112 for supporting surgical supplies, and a disposal station 114. The disposal station 114 may include some or all of a waste bin, an autoclave, a collection bin for items to be sanitized elsewhere, a hazardous material disposal bin, or other receptacle for storing or processing used surgical supplies.

One or more setup robots 116 may be positioned in the operating environment 100. In the illustrated embodiment, a single setup robot 116 is used. In use, the setup robot 116 prepares one station 102a, 102b while the other station 102b, 102a is in use. However, in other embodiments, a setup robot 116 is provided for each station 102a, 102b.

In the illustrated embodiment, the setup robot 116 includes a robotic arm 118. The robotic arm 118 may be a serial robotic arm and may have 4 to 8, or possibly more, degrees of freedom. The degrees of freedom may be sufficient to position the end effector 120 of the robotic arm 118 at arbitrary three-dimensional positions and orientations within the working envelope of the robotic arm 118. The degrees of freedom may include one or multiple degrees of freedom of a gripper included in the end effector 120, a tool changer, or other components including one or more degrees of freedom.

As shown in FIG. 1B, the end effector 120 may be a gripper for grasping and releasing objects. The end effector 120 may be any structure configured to selectively secured to and release objects. The end effector 120 may be configured to selectively secure to and release from objects with structures or features specifically designed to dock with the end effector 120. The end effector 120 may incorporate magnets, vacuum pads, or any other type of attachment structure for selectively securing to and releasing from objects.

In the illustrated embodiment, the robotic arm 118 is mounted to a rail 122 by a rail actuator 124. In other embodiments, the robotic arm 118 may be manually moved along the rail 122 and automatically or manually locked in position such that a rail actuator 124 is omitted. The rail actuator 124 includes a motor and a gear, wheel, or other structure engaging the rail 122 that is driven by the motor to move the base 126 of the robotic arm 118 to various positions along the rail 122. The rail 122 may be mounted to a floor, ceiling, or wall of the operating environment 100. Multiple rails and corresponding actuators may be used to implement a two- or three-dimensional translating gantry.

Precise positioning of the end effector 120 may be performed in various ways. In a first implementation, a kinematic state of the setup robot 116 and a known mapping of objects in the operating environment 100 is used along with obstacle detectors to position the end effector 120. In such embodiments, cameras or other local positioning system (LPS) is not used. In a second implementation, one or more camera 128 is mounted to the robotic arm 118 on or near (e.g., within 15 cm of and rigidly coupled to) the end effector 120. Images from the one or more cameras 128 may then be processed to determine the location and orientation of the end effector 120 and used as feedback to control the robotic arm 118. In a third implementation, cameras 130 are distributed around the operating environment and have the end effector 120 in a field of view thereof. The end effector 120 and possibly links and/or joints of the robotic arm 118 may have markings thereon to facilitate recognition thereof in images from the cameras 130. Images from the cameras 130 may then be processed to determine the position and orientation of the end effector 120 and used to control the robotic arm 118 to achieve a desired position and orientation of the end effector 120.

A supply area 132, e.g. table, may be positioned in the operating environment within the operating envelope of the setup robot 116. The supply area 132 may support a plurality of trays 134. Each tray is loaded with supplies for an ophthalmic treatment. In some scenarios, multiple trays 134 are used for a single ophthalmic treatment. The supply area 132 may include a gate 136 that allows trays 134 to drop, slide, or otherwise move into a pickup area 138. For example, trays 134 may be arranged according to a schedule of ophthalmic treatments such that each tray 134 may be retrieved by the setup robot 116 for each ophthalmic treatment in the schedule.

In use, while a surgeon 140 is operating on a patient 106 in station 102b, the setup robot 116 retrieves a tray 134 for a next ophthalmic treatment and loads the tray 134 onto the surgical table 112 of station 102a.

In some embodiments, each station 102a, 102b may include a console 144 providing ports for connecting tubes 146 for conducting vacuum pressure or infusion fluid, connecting electrical lines 148 for supplying power, or other types of ports. The setup robot may prepare a station 102a, 102b for a surgery by connecting each tube 146 and electrical line 148 between the console 144 (or other housing for a port) and an instrument, such as an instrument in the tray 134.

Referring to FIG. 1C, each tray 134 may include recesses 150. Some or all of the recesses 150 hold an item 152 for use during an ophthalmic treatment. Each item may be an instrument, a consumable product, a structure to be implanted, or other item for use during an ophthalmic treatment.

There are many items 152 that may be used to perform any number of ophthalmic treatments such as phacoemulsification and IOL placement, vitrectomy, glaucoma surgery, retinal attachment, refractive surgery (laser-assisted in situ keratomileusis (LASIK), small incision lenticule extraction (SMILE), implantable contact lens (ICL), etc.), or other ophthalmic treatments. A non-limiting list of possible items 152 includes the following:
A sideport incision instrument
Topical or injected anesthesia and corresponding syringe or other dispenser
Cystotome
Balanced salt solution (BSS)
Centurion handpiece
Silicone, metal, or polymer irrigation/aspiration (I/A) tip
Ophthalmic viscosurgical device
Tryphan blue and applicator
Forceps
OVD removal tool
Fluidics management system (FMS) pack
Metal or plastic handpiece
Pre-loaded disposable or reusable intraoptical lens (IOL) injector
Primary incision instrument
Irrigation/hydrodissection tool
Sutures
Drapes

In some embodiments, the layout of the tray 134 is known such that a controller of the setup robot 116 does not require visual recognition of the item within each recess 150. Instead, the controller may simply position the end effector at a known location of a recess 150 containing an item 152 and lift the item 152 from the tray. Recesses 150 and/or items 152 therein may further include markings, text, or other computer-readable symbols that may be used to identify the item 152 located in a particular recess 150.

Referring to FIG. 1D, items 152 may be positioned in the tray 134 bare, i.e., ready for use, or may be contained within a protective package. The setup robot 116 may therefore be configured to remove an item 152 from a corresponding package 154. For example, the setup robot 116 may include an extraction tool 156. The extraction tool 156 may include, for example, a grasping structure 158 to hold the package 154 and a cutting tool 160 configured to cut the package 154, such as the illustrated scissor blades, a single blade, or other type of cutting tool. Once cut open, the end effector 120 may then remove the item 152 from the package 154. Prior to or after cutting the package 154, the extraction tool 156 may invert the package 154 or orient the package 154 at an angle (e.g., 25 to 65 degrees, such as 45 degrees) with the cut end oriented downward to release the item 152 onto a surface for picking up by the end effector 120.

Other configurations are also possible. For example, items may be packaged within containers and the extraction tool 156 may be configured to interface with the containers by, for example, unscrewing a lid, pressing a button to release a lid, prying open a spring-loaded lid, inserting a pin to release the lid, or otherwise opening the container to permit access to an item 152 contained therein.

The package 154 and or item 152 may have a marking 162 facilitating the identification of the item 152 contained in the package 154 and possibly facilitating determination of the orientation of the package 154 from representations of the package 154 and marking 162 in images received from the cameras 128, 130.

Referring to FIG. 1E, some or all of the cameras 128, 130, setup robot 116, and extraction tool 156 may be connected by wires or wirelessly to a controller 172. The controller 172 may be implemented as a general-purpose computer, programmable logic controller (PLC), or other electronic device programmed to perform the functions ascribed herein to the controller 172.

The controller 172 may store or access a tray layout 174 for each tray 134 to be used for each ophthalmic treatment. The tray layout 174 may include an identifier of each tray 134 enabling the tray 134 to be identified, e.g., a marking, text, or other symbol affixed to the tray 134. The tray layout 174 may include a specification of the location of recesses 150 and an identifier of the item 152 positioned within each recess 150.

In some embodiments, to further facilitate the identification of items 152, the controller 172 may store or access an instrument library 176. For each type of item 152, the instrument library 176 may include such information as a marking, text, or other symbol that uniquely identifies the item 152, a position of each item 152 in each tray layout 174, a three-dimensional model of the item 152 enabling the items 152 to be identified in images from the cameras 128, 130, one or more two-dimensional images from different angles, or other data to facilitate machine identification of each type of item 152.

In use, the controller 172 may evaluate images from one or both of the cameras 128 and the cameras 130, determine that a tray 134 in the pickup area 138 is the correct tray for of a scheduled ophthalmic treatment by detecting identification data in the images, cause the setup robot 116 to grasp the tray 134 and move the tray 134 to the surgical table 112 of a station 102a, 102b for which the ophthalmic treatment is scheduled. As shown in FIG. 1A, the range of motion of the robotic arm 116 along the rail 122 enables the setup robot 116 to place a tray 132 on the surgical tables 112 of both stations 102a, 102b.

The controller 172 may further be programmed to identify used items 152 returned to the tray 132 in the images from one or both of the cameras 128, 130 and move the used items 152 to a disposal station 114 to be disposed of or disinfected for subsequent use.

In some embodiments, the controller 172 is programmed to grasp an item 152 from a tray 132 and pass the item 152 to the surgeon 140. For example, the controller 172 may be configured with voice commands 178. Voice commands may specify an action and an identifier of an item 152, e.g., action: pass, item identifier: intraocular lens insertion device. Possible actions may include to pass an item 152 from a tray 132 to a surgeon, receive an item 152 from a surgeon and pass the item 152 to the disposal station 114, or other actions described below. The controller 172 may be coupled to a microphone 180 present in the operating environment 100 and positioned to detect the voice commands of the surgeon 140. A single microphone 180 may be present or each station 102a, 102b may have a corresponding microphone 180.

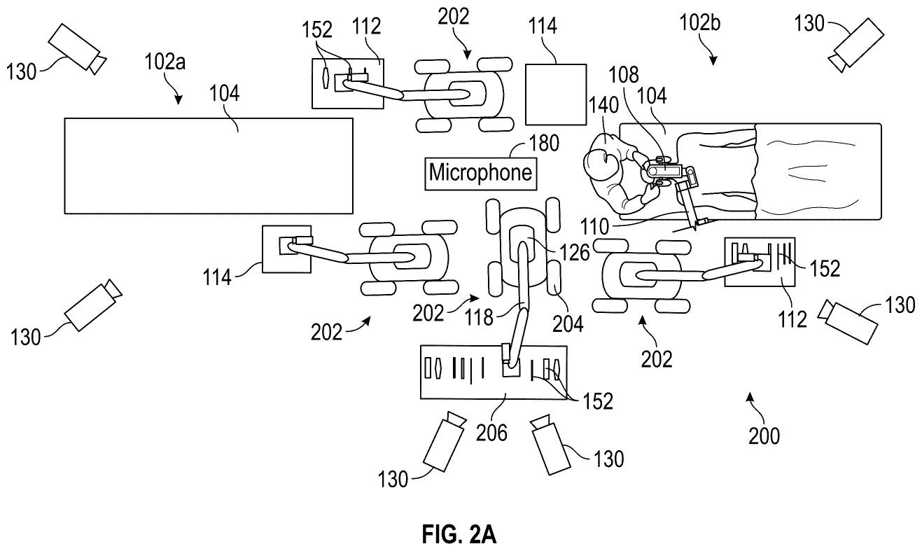

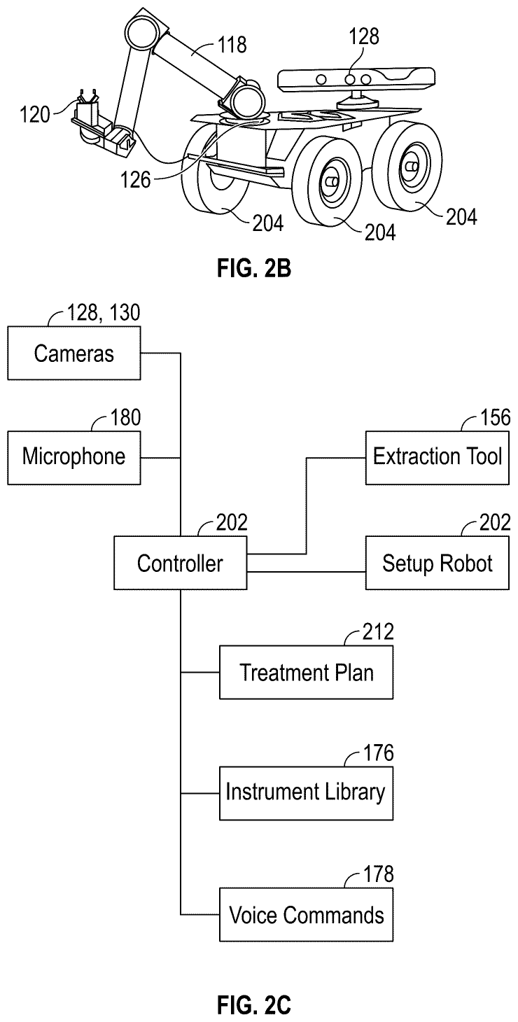

FIGS. 2A and 2B illustrates an alternative operating environment 200. The operating environment 200 may include stations 102a, 102b, patient supports 104, surgical microscope 108, support 110, surgical table 112, and one or more disposal stations 114, and cameras 128, 130 as described above.

In the operating environment 200, a setup robot 202 may be used. The setup robot 202 may include a robotic arm 118 as described above. The operating environment 200 may further include an extraction tool 156 as described above. The robotic arm 118 may include an end effector 120 and camera 128 as described above. The base 126 of the robotic arm 118 may be mounted to actuated floor engaging members 204 configured to move the base 126 in one or more dimensions along a floor of the operating environment 200. The floor engaging members 204 may be embodied as wheels, treads, articulated legs, or any other approach for inducing translational motion across a flat surface.

The setup robot 202 or the setup robot 116 as described above may be used without the benefit of pre-packed trays 132. Instead, images from the cameras 128, 130. For example, an end effector 120 and camera 128 of the robotic arm 118 may be used to identify, grasp, and place items 152 that are in a supply area 206 but not necessarily positioned in trays 132. The items 152 may be in bins with other items 152 of the same type. The items 152 may be in dispensers configured to interface with the end effector 120. The items 152 may also be laid out on a flat surface. Where a disposal station 114 is an autoclave or other type of cleaning device, the disposal station 114 may also function as a supply area 132 from which items 152 are retrieved using the setup robot 202 following cleaning and/or disinfection.

Referring to FIG. 2C, while still referring to FIG. 2A, a controller 210 may access a treatment plan 212 listing identifiers of items 152 to place on the surgical table 112 of a station 102a, 102b for an ophthalmic treatment represented by the treatment plan 212. The identifiers may reference the instrument library 176 as described above such that the controller 210 may access a model, images, markings, or other data enabling the controller 210 to identify representations of each item 152 in images from the cameras 128, 130. The controller 210 may therefore use the instrument library 176 to identify each item 152 listed in a treatment plan in images from the cameras 128, 130, cause the setup robot 202 to grasp each item 152 with the end effector 120, and transfer each item 152 to a surgical table 112 of a station 102a, 102b at which the ophthalmic treatment represented by the treatment plan 212 is scheduled. In a like manner, the controller 210 may instruct the setup robot 202 to transfer an item 152 to a disposal station 114.

As for the operating environment 100, the controller 210 may be coupled to a microphone 180 and detect and execute voice commands in the output of the microphone 180 according to voice commands 178 stored by or accessed by the controller 210 as described above.

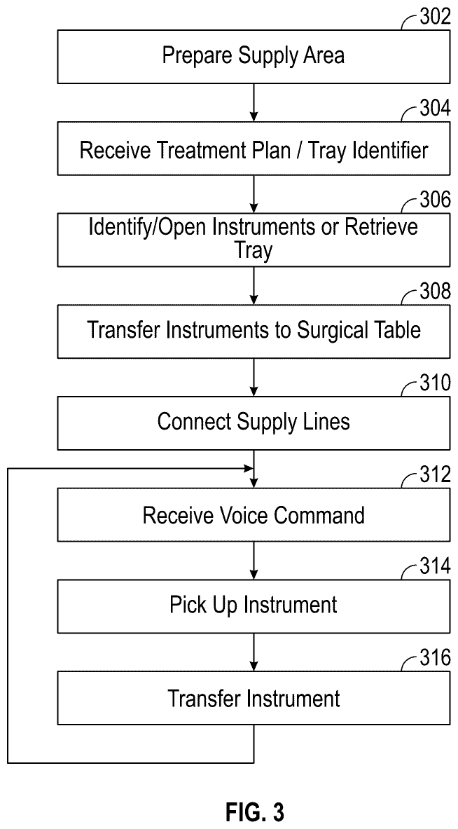

FIG. 3 illustrates a method 300 that may be executed by the controller 172 of the operating environment 100 or the controller 210 of the operating environment 200 with human actions as indicated in the description below.

The method 300 includes preparing, at step 302, a supply area 132, 206. For the operating environment 100, step 320 may include arranging one or more trays 134 with respect to the gate 136 to be distributed by the gate 136 to the pickup area 318. For the operating environment 200, step 320 may include arranging items 152 in the supply area 206 in bins, in prescribed locations, and/or in arbitrary locations with items 152 being identified through image analysis of images from the cameras 128, 130. Step 320 may be performed by a human, the setup robot 116, 202, or some other robot or other type of machine.

The method 300 may include receiving, at step 304, one of (a) a treatment plan 212 specifying identifiers of items 152 to be used in an ophthalmic treatment represented by the treatment plan 212 and (b) an identifier of a tray 134 containing items 152 to be used for an ophthalmic treatment. Step 304 may include receiving an identifier of a tray layout 174 or other data describing a tray layout 174, e.g., the location and sizes of recesses and identifiers of items 152 positioned within the recesses 150 of a tray 134.

For the operating environment 200, the method 300 may include identifying, at step 306, representations of items 152 identified in the treatment plan in images of a supply area 206 received from one or both of the cameras 128, 130. Step 306 may include using information provided in the instrument library 176 and associated with identifiers of items 152 included in the treatment plan 212. In embodiments where items 152 are in packages 154, step 306 may include opening the packages 154 and removing the items 152, such as with the extraction tool 156.

The method 300 may further include transferring, at step 308, the items identified at step 306 to the surgical table 112 of a station 102a, 102b scheduled for performance of an ophthalmic treatment represented by the treatment plan 212. Step 308 is performed by the setup robot 116, 202. Since the end effector 120 of the setup robot 116, 202 may only be capable of holding a single item 152 at a time, steps 306 and 308 may be performed repeatedly for each item 152 identified in the treatment plan 212. For the operating environment 100, step 308 includes transferring the tray 134 identified at step 304 and the items 152 contained thereon to the surgical table 112.

In some embodiments, the method 300 may include connecting, at step 310, supply lines (pneumatic tubes 146 and/or electrical lines 148) to one or more of the items 152. Step 310 may be performed using the setup robot 116, 202 or may be performed by a human operator. In some embodiments, the method 300 may end following step 308 or following step 310.

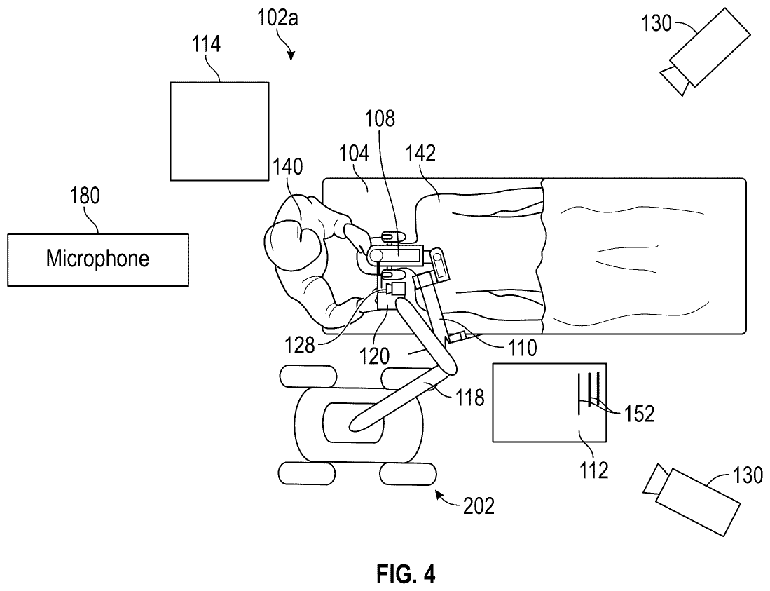

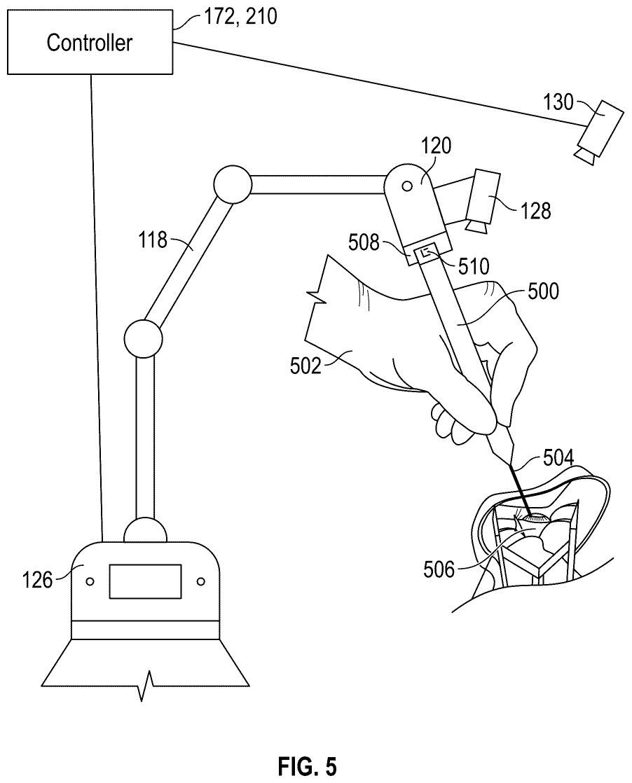

Referring to FIGS. 4 and 5, while continuing to refer to FIG. 3, in some embodiments, the setup robot 116, 202 may perform functions in addition to setting up a station 102a, 102b for performing an ophthalmic treatment.

For example, the method 300 may include receiving, at step 312, a voice command. For example, the microphone 180 may detect a surgeon 140 speaking a phrase. Step 312 may therefore include decoding a command and an identifier of an item 152 in the phrase. The method 300 may therefore include, at step 314, picking up the item 152, such as an instrument, referenced in the phrase using the end effector 120 and using the setup robot 116, 202 to transfer, at step 316, the item 152 to the surgeon 140, a disposal station 114, or other location specified by the command in the phrase. For example, as shown in FIG. 4, a the setup robot 116, 202 may pick up an item 152 from a surgical table 112 and transfer the item 152 to the hand of a surgeon 140.

In some instances, the setup robot 116, 202 may be in process of preparing a station 102a when a surgeon 140 performing an ophthalmic treatment in station 102b utters a voice command. Accordingly, the setup robot 116, 202 may interrupt preparation of the station, execute the voice command, and then return to setup of the station 102a.

Referring to FIG. 5, another voice command may instruct the controller 172, 210 to immobilize an instrument 500 held in the hand 502 of the surgeon 140. The surgeon 140 may instruct the controller 172, 210 to immobile the instrument 500 while a portion 504 of the instrument 500 is positioned within the eye 506 of the patient 142. For example, the instrument 500 may provide lighting or infusion fluid while the surgeon 140 uses another instrument. Alternatively, the surgeon 140 may need to rest momentarily or pause an ophthalmic treatment for another reason.

In response to the voice command, the controller 172, 210 may cause the end effector 120 to grasp the instrument 500. The controller 172, 210 may cause the end effector 120 without causing unacceptable movement of the instrument 500, e.g., translational or rotational movement greater than predefined thresholds. The end effector 120 may include a docking structure 508 configured to engage the instrument 500 smoothly and without causing unacceptable movement. Likewise, the instrument 500 may include markings 510 such that representations of the markings 510 may be detected in images from the cameras 128, 130 in order to precisely position the end effector 120 relative to the instrument 500 in order to avoid causing unacceptable movement of the instrument 500.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A system comprising:
one or more patient stations configured to facilitate performance of ophthalmic treatments;
a setup robot having the one or more patient stations in a range of motion thereof; and
a controller coupled to the setup robot and configured to cause the setup robot to prepare the one or more patient stations for the ophthalmic treatments.

2. The system of claim 1, wherein the one or more patient stations include a patient support.

3. The system of claim 1, wherein the one or more patient stations include one or more surgical microscopes.

4. The system of claim 1, wherein the one or more patient stations include a table for supporting items at least one of used or consumed during the ophthalmic treatments.

5. The system of claim 4, wherein the controller is configured to cause the setup robot to transfer the items from a supply area to the table of each patient station of the one or more patient stations.

6. The system of claim 5, wherein the items include at least one of supplies consumed during the ophthalmic treatments and instruments used during the ophthalmic treatments.

7. The system of claim 5, wherein the controller is configured to cause the setup robot to transfer trays loaded with the items to the table of each patient station of the one or more patient stations.

8. The system of claim 7, wherein each tray includes a plurality of recess configured to store the items.

9. The system of claim 5, wherein the controller is configured to cause the setup robot to individually grasp an item of the items and transfer the item to the table of a patient station of the one or more patient stations.

10. The system of claim 9, further comprising one or more cameras coupled to the controller, the controller configured to:
receive images from the one or more cameras; and
identify the items in the images from the one or more cameras.

11. The system of claim 4, further comprising one or more microphones coupled to the controller, the controller further configured to:
detect a phrase uttered by a surgeon in one or more outputs of the one or more microphones; and
cause the setup robot to execute a voice command included in the phrase.

12. The system of claim 11, wherein the controller further configured to:
detect an item identifier in the phrase; and
cause the setup robot to pick up and transfer an item of items referenced by the item identifier.

13. The system of claim 11, wherein the controller further configured to cause the setup robot to execute the voice command by immobilizing an item of the items held by the surgeon.

14. A method comprising:
instructing, by a controller device, a setup robot to prepare a first station for performing a first ophthalmic treatment with respect to a first patient in the first station; and
during the first ophthalmic treatment, instructing, by the controller device, the setup robot to prepare a second station for performing a second ophthalmic treatment with respect to a second patient.

15. The method of claim 14, further comprising:
transferring, by the setup robot, one or more items to a table in the first station, the one or more items including at least one of materials consumed during the first ophthalmic treatment or instruments used during the first ophthalmic treatment.

16. The method of claim 15, wherein transferring the one or more items to the table comprises transferring a tray containing the one or more items.

17. The method of claim 15, further comprising:
detecting, by the controller device, a command from a surgeon; and
in response to detecting the command, instructing, by the controller device, the setup robot to transfer an item of the one or more items from the table to a hand of a surgeon.

18. The method of claim 17, further comprising:
in response to the command, instructing, by the controller device, the setup robot to suspend preparation of the second station.

19. The method of claim 14, further comprising:
detecting, by the controller device, a command from a surgeon; and
in response to detecting the command, instructing, by the controller device, the setup robot to immobilize an instrument held in a hand of a surgeon.

20. The method of claim 19, wherein the instrument includes a portion inserted within an eye of the first patient.
